(19) United States
(12) Patent Application Publication
(10) Pub. No.: US 2024/0092796 A1
(43) Pub. Date: Mar. 21, 2024

(54) PRODUCT OF INTERNAL DEHYDRATION OF HIGH-PURITY SORBITOL

(71) Applicant: ROQUETTE FRERES, Lestrem (FR)

(72) Inventors: Nicolas JACQUEL, Pontarlier (FR); René SAINT-LOUP, Lomme (FR); Théodore VANBESIEN, Armentières (FR)

(21) Appl. No.: 18/254,320

(22) PCT Filed: Nov. 19, 2021
(86) PCT No.: PCT/EP2021/025454

§ 371 Date: May 24, 2023

(30) Foreign Application Priority Data
Nov. 26, 2020 (FR) 2012215

(51) Int. Cl. C07D 493/04

(52) U.S. Cl. CPC C07D 493/04

(57) ABSTRACT

The present invention relates to a product of internal dehydration of sorbitol, characterized in that it has a total residual nitrogen atom content of between 0.01 ppm and 150 ppm, preferably of between 0.02 ppm and 20 ppm, more preferably of between 0.05 ppm and 10 ppm, and, more preferentially, of between 0.07 ppm and 5 ppm, this residual content being expressed as dry weight relative to the total dry weight of said product, and in that it has a total residual sulphur atom content of between 0.0001 ppm and 100 ppm, preferably between 0.0002 ppm and 50 ppm, more preferably between 0.0004 ppm and 30 ppm, and more preferentially, between 0.0008 ppm and 20 ppm, this total residual content being expressed in dry weight relative to the dry weight of said product; a method for purifying such a product and a polymer comprising a unit corresponding to said product.

The present invention relates to a product of internal dehydration of high-purity sorbitol, a method for manufacturing such a product and a polymer comprising said product as a monomer. Most particularly, the present invention relates to a high-purity isosorbide, a method for manufacturing such isosorbide and a polymer comprising isosorbide as a monomer.

### PRIOR ART

Anhydrous sugar alcohols, in particular sorbitol derivatives, are known for their applications and uses in various industries. Isosorbide, 1,4:3,6-dianhydrosorbitol, a product of internal dehydration of sorbitol, is of major interest as a recoverable natural resource in the manufacture of polymers. Isosorbide is indeed a sorbitol derivative that can be obtained from various natural resources, including corn starch and cassava (tapioca).

With regard to the uses of anhydrous sugar alcohols, the requirements of purity depend on the intended application. In food and therapeutic applications, for example, it is essential that the compounds containing it do not include any impurities that could be harmful to the individual or to the organism that uses them. For the preparation of polymers, in particular polymers which require optical transparency such as those used in packaging, a requirement in terms of purity of the monomer is that no material or impurity must be present in the monomer which could result in an unacceptable degree of coloration of the polymer during its synthesis and/or its transformation. During the transformation of said alcohols, more particularly during the synthesis of polymers using isosorbide as a monomer require high temperatures, isosorbide can develop a coloration originating because of the presence of impurities therein. Thus, the coloring of the final product is no longer controlled. Such a coloring is therefore not desired.

Several methods for purifying anhydrous sugar alcohols are documented in the art. The purification of these alcohols may, for example, involve a recrystallization step in aliphatic alcohols, as described in document WO0041985.

Document WO 2008143269 describes a method for obtaining a polycarbonate based on isosorbide and a carbonic acid diester wherein the synthesis of said polymer is followed by a distillation step so as to remove the phenol formed. The polycarbonate thus obtained has a residual content of Na, Fe and Ca less than 2 ppm.

In order to retain a cation level present in an alcohol of anhydrous sugar below 1 ppm, document KR101736182 describes a method for purifying such an alcohol comprising a passage over a cation exchange resin, the pH of the solution comprising said alcohol to be purified being adjusted to at least 5, for example between 5 and 8, at room temperature.

Document KR101736180 describes a method for purifying an anhydrous sugar alcohol wherein the formic acid content is less than 1 ppm. This method comprises a passage over a strong base anion exchange resin.

Document EP1882712 relates to a polyester obtained from a diol and a carboxylic acid wherein both the content of impurities and the number of terminal acid groups are reduced so as to reduce the hydrolysis and therefore to improve the stability of the polyester over time. To do this, the content of sulphur atoms in the monomers is between 0.01 ppm and 100 ppm, the content of nitrogen atoms in the monomers is between 0.01 ppm and 2000 ppm and the number of end acid groups in the polyester is less than 50 equivalents/metric ton.

Document FR2810040 relates to a method for purifying a composition wherein the composition to be purified is successively subjected to ion exchange and discoloration.

Today, as indicated previously, in the many applications of isosorbide, the purity plays a crucial role on the quality of the products ultimately obtained. In particular, the applicant has demonstrated the particularly large impact of certain elements: nitrogen, sulphur, sodium, calcium, potassium and magnesium.

According to the Applicant, it also has not been possible until now in industrial practice is to efficiently prepare internal dehydration products of sorbitol, for example isosorbide, simultaneously having a nitrogen content and very low sulphur.

The applicant company has found, after numerous research, that it was possible to obtain internal dehydration products of a higher-purity sorbitol that can then be used during the manufacture of polymers having very satisfactory optical properties, in particular in terms of their coloring and their lightness while maintaining good viscosity and thermal resistance characteristics.

### SUMMARY OF THE INVENTION

According to a first object, the present invention relates to a product of internal dehydration of sorbitol, characterized in that it has a total residual nitrogen atom content of between 0.01 ppm and 150 ppm, preferably of between 0.02 ppm and 20 ppm, more preferably of between 0.05 ppm and 10 ppm, and, more preferentially, of between 0.07 ppm and 5 ppm, this residual content being expressed as dry weight relative to the total dry weight of said product, and in that it has a total residual sulphur atom content of between 0.0001 ppm and 100 ppm, preferably between 0.0002 ppm and 50 ppm, more preferably between 0.0004 ppm and 30 ppm, and more preferentially, between 0.0008 ppm and 20 ppm, this total residual content being expressed in dry weight relative to the dry weight of said product.

According to a second object, the invention relates to a method for purifying a product of internal dehydration of sorbitol according to the first object, said method comprising a succession of steps:
a) a step of supplying said product of internal dehydration of sorbitol,
b) a step of distilling said dehydration product so as to form a distillation product A,
c) a step of dissolving in water said distillation product A with the addition of a basic compound so as to form a solution B,
d) at least one step of discoloration of solution B resulting from the step of redissolving with addition of a basic compound,
e) at least one step of ion exchange of the solution resulting from the bleaching step, and
f) a step of recovering the resulting purified product C, said basic compound being added in an amount of between 1 and 6 g, preferably between 2 and 5 g per Kg of product of internal dehydration of sorbitol provided in step a).

According to a third object, the invention relates to a polymer selected from a polyester, a polycarbonate, a polyarylether, a polyurethane or a polyepoxide, said polymer is characterized in that it comprises a unit corresponding to the product of internal dehydration of sorbitol according to the first object or obtained from a method according to the second object.

Products of internal dehydration of sorbitol according to the invention have an excellent degree of purity, most particularly products having both a very low content of sulphur and nitrogen.

The method according to the invention therefore makes it possible to obtain such products of internal dehydration of sorbitol having excellent purity while using conventional purification techniques.

The polymers obtained based on products of internal dehydration of sorbitol according to the invention have remarkable optical properties in terms of coloring and lightness, without affecting the other essential characteristics in the field of plastic objects, such as viscosity and thermal resistance

### DETAILED DESCRIPTION OF THE INVENTION

A first object of the invention relates to a product of internal dehydration of sorbitol having a total residual nitrogen atom content of between 0.01 ppm and 150 ppm, preferably of between 0.02 ppm and 20 ppm, more preferably of between 0.05 ppm and 10 ppm, and, more preferentially, of between 0.07 ppm and 5 ppm, this residual content being expressed as dry weight relative to the total dry weight of said product, and having a total residual sulphur atom content of between 0.0001 ppm and 100 ppm, preferably between 0.0002 ppm and 50 ppm, more preferably between 0.0004 ppm and 30 ppm, and more preferentially, between 0.0008 ppm and 20 ppm, this total residual content being expressed in dry weight relative to the dry weight of said product.

“Product of internal dehydration of sorbitol” is understood to mean any product or composition resulting, in any way, in one or more steps, from the removal of one or more water molecules from the original internal structure of sorbitol.

It may advantageously be an product of internal dehydration of sorbitol, such as a composition of isosorbide (1,4-3,6 dianhydro sorbitol).

According to one embodiment, the product of internal dehydration of sorbitol has a total residual content of sodium and potassium atoms of between 0.002 ppm and 100 ppm, preferably between 0.004 ppm and 50 ppm, more preferably between 0.006 ppm and 20 ppm, and, more preferentially, between 0.008 ppm and 10 ppm, this total residual content being expressed in dry weight relative to the total dry weight of said product.

Residual content of sodium and potassium atom is understood to mean the residual content of all of both atoms at the same time.

According to one embodiment, the product of internal dehydration of sorbitol has a total residual content of calcium and magnesium atoms of between 0.005 ppm and 100 ppm, preferably between 0.010 ppm and 50 ppm, more preferably between 0.015 ppm and 20 ppm, and, more preferentially, between 0.020 ppm and 10 ppm, this total residual content being expressed in dry weight relative to the total dry weight of said product.

Residual content of calcium and magnesium atoms is understood to mean the residual content of all of both atoms at the same time.

According to one embodiment, the product of internal dehydration of sorbitol has a total residual content of iron atoms of between 0.005 ppm and 100 ppm, preferably between 0.010 ppm and 50, more preferably between 0.015 ppm and 20 ppm, and, more preferentially, between 0.020 ppm and 10 ppm, this total residual content being expressed in dry weight relative to the total dry weight of said product.

According to one embodiment, the product of internal dehydration of sorbitol has a total residual content of chlorine atoms of between 0.005 ppm and 100 ppm, preferably between 0.010 ppm and 50, more preferably between 0.015 ppm and 20 ppm, and, more preferentially, between 0.020 ppm and 10 ppm, this total residual content being expressed in dry weight relative to the total dry weight of said product.

The internal dehydration products of sorbitol according to the invention correspond to products or compositions as defined above, the dehydration possibly being total or partial.

Given their purity characteristics, these internal dehydration products of sorbitol can be used advantageously in numerous industries and in particular as a synthetic intermediate, comonomer (including chain extender), solvent agent, plasticizing agent, lubricating agent, bulking agent, sweetener and/or active ingredient, in the preparation of polymeric or nonpolymeric products or mixtures, biodegradable or not, intended for the chemical, pharmaceutical, cosmetic or food industries.

A second object of the invention relates to a method for purifying a product of internal dehydration of sorbitol according to the first object, said method comprising a succession of steps:
a) a step of supplying said product of internal dehydration of sorbitol,
b) a step of distilling said dehydration product so as to form a distillation product A,
c) a step of dissolving in water said distillation product A with the addition of a basic compound so as to form a solution B,
d) at least one step of discoloration of solution B resulting from the step of redissolving with addition of a basic compound,
e) at least one step of ion exchange of the solution resulting from the bleaching step, and
f) a step of recovering the resulting purified product C, said basic compound being added in an amount of between 1 and 6 g, preferably between 2 and 5 g per Kg of product of internal dehydration of sorbitol provided in step a).

Preferably, the distillation step is carried out in a continuous evaporator. Such a device, for example of the falling-flow type or better yet, of the wiped-film or short-path type, makes it possible to limit the temperatures and residence time to which the reaction raw materials are thus subjected.

The intermediate pH of the distillation product A can be measured.

The distillation product A is dissolved in water so as to obtain an aqueous solution comprising between 50 and 90% dry matter, preferably between 60 and 80% dry matter. Once the solution has been obtained, a basic compound is added with stirring at 150 rotations per minute (RPM) and at ambient temperature (20° C.). The medium thus obtained can be kept under stirring for a period of between 30 minutes and two hours, preferably between 45 minutes and 75 minutes.

The medium thus obtained may be subjected to a filtration step.

The filtrate can then be diluted in water so as to obtain an aqueous solution comprising between 30 and 70% dry matter, preferably between 40 and 60% dry matter.

The pH of solution B can be measured.

According to one embodiment, the pH of solution B is between 4 and 10, preferably between 7 and 9.

According to one embodiment, the basic compound is chosen from alkaline earth hydroxides such as magnesium hydroxide, calcium hydroxide, strontium hydroxide or barium hydroxide, preferably calcium hydroxide.

According to one embodiment, the treatment by the discoloring step comprises at least one passage over a column of granular activated carbon.

According to one embodiment, at least one ion exchange step is chosen from a passage on a cation exchange resin or a passage on an anion exchange resin or a mixture of two, preferably the cation exchange resin is a strong cation exchange resin and the anion exchange resin is a strong anion exchange resin.

Preferably, if the method comprises at least two ion exchange steps, they will follow one another so that the solution is recovered and passed successively on a cation exchange resin column then an anion exchange resin column.

More preferably, if the method comprises at least two ion exchange steps, they will follow one another so that the solution is recovered and passed successively on a strong cation exchange resin column then a strong anion exchange resin column.

The product of internal dehydration of sorbitol used according to the purification method above corresponds to a single product or to a composition comprising a mixture of entities derived from the internal sorbitol dehydration reaction.

According to one embodiment, the method is free of an additional discoloration step after the ion exchange step and before the step of recovering the resulting product.

According to one embodiment, the method is free of an additional recrystallization step of the different intermediate products of said method.

According to one embodiment, the method for purifying an product of internal dehydration of sorbitol according to the first object, said method consisting of a succession of steps of:
a) a step of providing said product of internal dehydration of sorbitol,
b) a step of distillation of said dehydration product so as to form a distillation product A,
c) a step of dissolving said distillation product A in water with addition of a basic compound so as to form a solution B,
d) at least one step of discoloration of the solution B resulting from the step of redissolving with addition of a basic compound,
e) at least one step of ion exchange of the solution resulting from the discoloration step, and
f) a step of recovering the resulting purified product C, said basic compound being added in an amount of between 1 and 6 g, preferably between 2 and 5 g per Kg of product of internal dehydration of sorbitol provided in step a).

A third object of the invention relates to a polymer selected from a polyester, a polycarbonate, a polyarylether, a polyurethane or a polyepoxide, said polymer is characterized in that it comprises a unit corresponding to the product of internal dehydration of sorbitol according to the first object or obtained from a method according to the second object.

The present invention will be described in more detail by means of the following examples which are in no way limiting.

### Examples

### Example 1: Synthesis of Isosorbide I1

1 Kg of sorbitol solution at 80% dry weight and 8 g of concentrated sulphuric acid are introduced under stirring to a dual-jacketed reactor. The mixture obtained is heated to 145° C. under vacuum (100 mbar) for 5 hours so as to remove the water contained in the reaction medium and the water from the dehydration reaction by distillation.

The crude reaction product is then cooled to 100° C. and then neutralized with 13.7 g of a 50% sodium hydroxide solution.

The isosorbide composition obtained is then distilled under vacuum using a wiped film evaporator in short path configuration. The pH of distilled isosorbide (in solution at 40% dry matter) is then 3.5.

The distillate is recovered and then redissolved in water in order to obtain a 70% dry matter solution. In this solution, 2.5 g of calcium hydroxide are added with vigorous stirring and at temperature. The medium is stirred for 1H.

The medium is then cloudy and opaque. The medium is then filtered on a Becko filter (0.45 μm) in order to obtain a clear solution. Water is then added in order to obtain a 50% DM solution. The pH of the final solution is 8.5.

This solution is then percolated on a column packed with granular activated carbon at a rate of 0.5 VV:H (volume of solution per fixed bed volume and per hour).

The solution is then recovered and passed successively on a strong cation exchange resin column and then a strong anion exchange resin column. The solution is then concentrated under vacuum to obtain, after crystallization and milling of the solid, a white powder.

### Example 2: Synthesis of Isosorbide I2

1 Kg of sorbitol solution at 80% dry weight and 8 g of concentrated sulphuric acid are introduced under stirring to a dual-jacketed reactor. The mixture obtained is heated to 145° C. under vacuum (100 mbar) for 5 hours so as to remove the water contained in the reaction medium and the water from the dehydration reaction by distillation.

The crude reaction product is then cooled to 100° C. and then neutralized with 13.7 g of a 50% sodium hydroxide solution.

The isosorbide composition obtained is then distilled under vacuum using a wiped film evaporator in short path configuration.

The distilled isosorbide is redissolved in distilled water in order to form a 50% dry matter solution. The pH of this solution is 3.5

This solution is then percolated on a column packed with granular activated carbon at a rate of 0.5 VV:H.

The solution is then recovered and passed successively on a strong cation exchange resin column and then a strong anion exchange resin column.

The solution is then concentrated under vacuum to obtain, after crystallization and milling of the solid, a white powder.

During this synthesis, the basic compound was not added during the step of dissolving the distillation product.

### Example 3: Synthesis of Isosorbide I3

1 Kg of sorbitol solution at 80% dry weight and 8 g of concentrated sulphuric acid are introduced under stirring to a dual-jacketed reactor. The mixture obtained is heated to 145° C. under vacuum (100 mbar) for 5 hours so as to remove the water contained in the reaction medium and the water from the dehydration reaction by distillation.

The crude reaction product is then cooled to 100° C. and then neutralized with 13.7 g of a 50% sodium hydroxide solution.

The isosorbide composition obtained is then distilled under vacuum using a wiped film evaporator in short path configuration.

The distillate is recovered and then redissolved in water in order to obtain a 70% dry matter solution. In this solution, 3 g of magnesium carbonate are added with vigorous stirring and at ambient temperature. The medium is stirred for 1 H. The solution being slightly cloudy, the medium is filtered on a Becko filter (0.45 μm)

Water is then added in order to obtain a 50% DM solution. The pH of the final solution is 9.5.

The distilled isosorbide is redissolved in distilled water in order to form a 50% dry matter solution.

This solution is then percolated on a column packed with granular activated carbon at a rate of 0.5 VV:H followed by treatment with a black powder at the height of 2% by mass of black relative to the dry matter. The solution is then filtered to recover the isosorbide solution.

The solution is then concentrated under vacuum to obtain, after crystallization and milling of the solid, a white powder.

### Example 4: Synthesis of Isosorbide I4

1 Kg of sorbitol solution at 80% dry weight and 8 g of concentrated sulphuric acid are introduced under stirring to a dual-jacketed reactor. The mixture obtained is heated to 145° C. under vacuum (100 mbar) for 5 hours so as to remove the water contained in the reaction medium and the water from the dehydration reaction by distillation.

The crude reaction product is then cooled to 100° C. and then neutralized with 13.7 g of a 50% sodium hydroxide solution.

The isosorbide composition obtained is then distilled under vacuum using a wiped film evaporator in short path configuration.

The distillate is recovered and then redissolved in water in order to obtain a 70% dry matter solution. In this solution, 9 g of a tetraethyl ammonium hydroxide solution (aqueous solution at 35% dry matter) are added with stirring and at ambient temperature. The medium is stirred for 1 H. The solution is clear after this treatment.

Water is then added in order to obtain a 50% dry matter solution. The pH of the final solution is 11.

The distilled isosorbide is redissolved in distilled water in order to form a 50% dry matter solution.

This solution is then percolated on a column packed with granular activated carbon at a rate of 0.5 VV:H followed by treatment with a black powder at the height of 2% by mass of black relative to the dry matter. The solution is then filtered to recover the isosorbide solution.

The solution is then concentrated under vacuum to obtain, after crystallization and milling of the solid, a white powder.

The isosorbides produced are respectively denoted 11, 12 and 13. The quantities of nitrogen, sulphur, sodium and potassium, magnesium, iron, chlorine and calcium are shown in Table 1.

These elements are assayed by inductively coupled plasma-atomic emission spectroscopy (ICP AES).

TABLE 1

| | Ex I1 | CEx I2 | CEx I3 | CEx I4 |
|---|---|---|---|---|
| Nitrogen (ppm) | 0.01 | 0.01 | 0.01 | 1100 |
| Sulphur (ppm) | 0.0001 | 100 | 103 | 95 |
| Sodium and potassium | 0.0001 | 65 | 74 | 125 |
| (ppm) | | | | |
| Magnesium (ppm) | 0.001 | 0.1 | 98 | 0.1 |
| Iron (ppm) | 0.001 | 95 | 75 | 50 |
| Chlorine (ppm) | 0.001 | 125 | 100 | 105 |
| Calcium (ppm) | 0.002 | 51 | 0.001 | 0.001 |

### Example 5: PEI30T Polyesters Based on Isosorbide I1 According to Example 1

893 g (14.4 mol) ethylene glycol, 700 g (4.8 mol) isosorbide I1, 2656 g (16 mol) terephthalic acid, 0.70 g Irganox 1010 and 0.70 g Hostanox P-EPQ (antioxidant), and 0.9820 g germanium dioxide (catalyst) are added to a 7 L reactor. To extract the residual oxygen from the isosorbide crystals, four vacuum-nitrogen cycles are performed once the temperature of the reaction medium is between 60 and 80° C.

The reaction mixture is then heated to 250° C. (4° C./min) under 2.5 bar of pressure and under constant stirring (150 rpm). The degree of esterification is estimated based on the amount of distillate collected. The pressure is then reduced to 0.7 mbar over 90 minutes according to a logarithmic gradient and the temperature is brought to 265° C.

These low-pressure and temperature conditions were maintained until an increase in torque of 19.8 Nm with respect to the initial torque was obtained.

Finally, a polymer rod is cast via the bottom valve of the reactor, cooled in a heat-regulated water bath at 15° C. and chopped up in the form of granules of approximately 15 mg.

Using such a method makes it possible to avoid contact between the heated polymer and oxygen, so as to reduce the coloration and the thermo-oxidative degradation.

The resin thus obtained has a reduced viscosity in solution of 60.5 mL/g. 1H NMR analysis of the polyester P1 shows that it contains 30.4 mol % of isosorbide relative to the diols.

The diethylene glycol unit content is 2.3 mol %.

The polymer is amorphous, and has a Tg of 112.4° C.

The coloration of the polymer measured on the granules is the following L*=60.8, a*=0.1, b*=3.9.

The haze measured on injected plates with a thickness of 2 mm is 2.8.

### Example 6: Example PE130T Polyesters Based on Isosorbide I2 According to Example 2

The protocol of example 5 is reproduced with the isosorbide of 12.

The resin obtained has a reduced viscosity in solution of 61.2 mL/g.

1H NMR analysis of the polyester P2 shows that it contains 29.9 mol % of isosorbide relative to the diols.

The diethylene glycol unit content is 2.5 mol %. The polymer is amorphous, and has a Tg of 112.1° C.

The coloration of the polymer measured on the granules is the following L*=55.4, a*=0.2, b*=7.2.

The haze measured on injected plates with a thickness of 2 mm is 5.1.

### Example 7: Example PE130T Polyesters Based on Isosorbide I3 According to Example 3

The protocol of example 5 is reproduced with the isosorbide of 13.

The resin obtained has a reduced viscosity in solution of 60.8 mL/g.

1H NMR analysis of the polyester P3 shows that it contains 30.5 mol % of isosorbide relative to the diols.

The diethylene glycol unit content is 2.3 mol %.

The polymer is amorphous, and has a Tg of 113.0° C.

The coloration of the polymer measured on the granules is the following L*=53.4, a*=0.3, b*=6.9.

The haze measured on injected plates with a thickness of 2 mm is 4.8.

### Comparative Example 8: Example PE130T Polyesters Based on Isosorbide I4 According to Example 4

The protocol of example EXP1 is reproduced with the isosorbide of 14.

The resin obtained has a reduced viscosity in solution of 61.8 mL/g.

1H NMR analysis of the polyester P3 shows that it contains 30.1 mol % of isosorbide relative to the diols.

The diethylene glycol unit content is 2.3 mol %.

The polymer is amorphous, and has a Tg of 111.3° C.

The coloration of the polymer measured on the granules is the following L*=54.1, a*=0.3, b*=7.5.

The haze measured on injected plates with a thickness of 2 mm is 5.6. The results of examples 5 to 8 are listed in Table 2 below.

TABLE 2

| | VISCOSITY | % ISB | TG | L* | a* | b* | haze |
|---|---|---|---|---|---|---|---|
| PEI30T I1 | 60.5 | 30.4 | 112.4 | 60.8 | 0.1 | 3.9 | 2.8 |
| PEI30T I2 | 61.2 | 29.9 | 112.1 | 55.4 | 0.2 | 7.2 | 5.1 |
| PEI30T I3 | 60.8 | 30.5 | 113 | 53.4 | 0.3 | 6.9 | 4.8 |
| PEI30T I4 | 61.8 | 30.1 | 111.3 | 54.1 | 0.3 | 7.5 | 5.6 |

From the results obtained in examples 5 to 8, the values of the parameter b* and of the haze of the polyesters based on the isosorbide according to the invention (11) are the lowest. Polyesters based on isosorbide according to the invention therefore have more satisfactory coloring and lightness.

### Example 9: Example Polycarbonate Based on Isosorbide I1 According to Example 1

1040 g (4.86 mol) of diphenyl carbonate, 502 g (3.44 mol) of isosorbide I1, 213 g (1.48 mol) of 1,4-cyclohexanedimethanol, 420 mg of Irganox 1010 (antioxidant) and 420 mg of Hostanox PEPQ (antioxidants), 6.1 mg of cesium carbonate (catalyst) are added to a 3 L reactor. To extract the residual oxygen from the isosorbide crystals, four vacuum-nitrogen cycles are performed once the temperature of the reaction medium is between 60 and 80° C.

The distillation column is heated at 110° C. to prevent crystallization of the phenol which is released during the reaction. The stirring speed is adjusted to 120 rpm (this will be reduced as the viscosity will increase). The reactor is then heated and a vacuum ramp is applied while increasing the temperature of the reaction medium. The temperature and pressure conditions used are as follows:
Heating to 150° C. at 800 mbar for 15 min
Heating from 150 to 190° C. while decreasing from 800 to 100 mbar in 45 min
Heating from 190 to 220° C. while decreasing pressure from 100 to 60 mbar in 45 min
decreasing the pressure from 60 to 10 mbar at 220° C. in 30 min.

After these 30 minutes, the torque is 22.6 Nm for stirring at 50 rpm.

A polymer rod is cast via the bottom valve of the reactor, cooled to 15° C. in a heat-regulated water bath and chopped up in the form of granules of approximately 15 mg.

The resin thus obtained has a reduced viscosity in solution of 52.5 mL/g.

1H NMR analysis of the polycarbonate P4 shows that it contains 74.2 mol % of isosorbide relative to the diols.

The polymer is amorphous, and has a Tg of 130.4° C.

The coloration of the polymer measured on the granules is the following L*=71.8, a*=0.0, b*=5.4.

The haze measured on injected plates with a thickness of 2 mm is 1.4.

### Example 10: Example Polycarbonate Based on Isosorbide I3 According to Example 3

The protocol of example 9 is reproduced, this time with the isosorbide of I3. The resin thus obtained has a reduced viscosity in solution of 49.4 mL/g.

1H NMR analysis of the polycarbonate P5 shows that it contains 70.1 mol % of isosorbide relative to the diols.

The polymer is amorphous, and has a Tg of 126.0° C.

The coloration of the polymer measured on the granules is the following L*=63.7, a*=−0.1, b*=8.7.

The haze measured on injected plates with a thickness of 2 mm is 4.1.

The results of examples 9 to 10 are listed in Table 3 below.

TABLE 3

| | VISCOSITY | % ISB | TG | L* | a* | b* | haze |
|---|---|---|---|---|---|---|---|
| POLYCARBONATE I1 | 52.5 | 74.2 | 130.4 | 71.8 | 0.0 | 5.4 | 1.4 |
| POLYCARBONATE I3 | 49.4 | 70.1 | 126 | 63.7 | −0.1 | 8.7 | 4.1 |

From the results obtained in examples 9 to 10, the values of the parameter b* and of the haze of the polycarbonates based on the isosorbide according to the invention (11) are the lowest. Polycarbonates based on isosorbide according to the invention therefore have more satisfactory coloring and lightness.

### Example 11: Example Polysulfone Based on Isosorbide I1 According to Example 1

2.92 g (0.020 mol, 1 eq) of isosorbide I1 (placed in a desiccator beforehand to eliminate residual water), 5.08 g (0.020 mol, 1 eq) of difluorodiphenyl sulfone and 5.58 g (0.040 mol, 2 eq) of K2CO3 are solubilized in 18.7 g of DMSO in a three-necked flask equipped with a gooseneck, a stirrer blade and a nitrogen inlet. The round-bottomed flask is heated to 140° C. using an oil bath for 20 hours. At the end of the reaction, 15 mL of DMSO are added to dilute the medium. The reaction medium is then precipitated in the form of threads in 1,000 mL of water, Büchner filtered, and then dried with an oven under vacuum.

The polysulfone P6 thus obtained has a reduced viscosity in solution of 36.1 mL/g The polymer is amorphous and has a Tg of 236.5° C.

The polymer was then formed as a film by solvent evaporation method from a polymer solution at 20 w % in DMSO. The viscous polymer solution was applied with a metal blade on a glass substrate. The deposition is then evaporated slowly in an oven following the following protocol: 50° C. for 16 h, 80° C. for 1 h, 130° C. for 1 h, 130° C. for 1 h and 180° C. for 2 hours.

At the end, a film with a thickness of approximately 100 microns is obtained. The film is colorless and has a haze of 0.2.

### Example 12: Example Polysulfone Based on Isosorbide I2 According to Example 2

The protocol of example 11 is reproduced, this time with the isosorbide of I2.

The polysulfone P7 thus obtained has a reduced viscosity in solution of 35.8 mL/g The polymer is amorphous and has a Tg of 236.2° C.

A 100-micron film produced according to the same procedure as in example 11 is slightly yellow and has a haze of 1.1.

From the results obtained in examples 11 to 12, the values of the haze of the polysulfones based on the isosorbide according to the invention (11) are the lowest. Polysulfones based on isosorbide according to the invention therefore have more satisfactory lightness.

The results of examples 11 and 12 are presented in Table 4.

TABLE 4

| | VISCOSITY | % ISB | TG | L* | a* | b* | haze |
|---|---|---|---|---|---|---|---|
| POLYSULFONE I1 | 36.1 | ? | 236.5 | / | / | / | 0.2 |
| POLYSULFONE I2 | 49.4 | 70.1 | 126 | / | / | / | 1.1 |

### Example 13: Example Isosorbide Diester D1 Based on Isosorbide I1 According to Example 1

In a dual-jacketed reactor, 3.04 Kg of caprylic acid (C8 linear saturated fatty acid) are added under stirring, followed by 1.4 Kg of isosorbide I1 (fatty acid/isosorbide molar ratio: 2.2). 30 g of methanesulfonic acid and 8.4 g of hypophosphorous acid are then added.

The reactor is heated to a set temperature of 160° C. and a vacuum of 100 mbar is applied to the system. Once the medium is at 90° C. and the first drops of water have been distilled, a vacuum ramp of 1000 to 30 mbar is carried out on 5 hours. Once the ramp is finished, the temperature setpoint of the reactor is brought to 170° C. for a duration of 2 h at 30 mbar.

Once esterification is complete, the heat is cut off and the medium is brought back to a temperature of 115° C. 15 mL of a 50% sodium hydroxide solution are then added to neutralize the catalysts. The reaction medium is allowed to cool to room temperature.

The excess fatty acid used is distilled on a wiped-film evaporator. The diester is recovered at the bottom of the tank that is taut where the excess acid is distilled.

The measurement of the coloration according to the APHA scale is carried out on a Loviond PFX-i Series spectrophotometer according to the ASTM D-1209 method (January 2005), with a rectangular tank of 5 cm in APHA color scale by a suitable colorimeter on the product without dissolution in any solvent.

The results are presented in Table 5.

TABLE 5

| Acid index | 1.0 mgKOH/g |
|---|---|
| Free fatty acid | 0.1% |
| Diester | 4.3% |
| Isosorbide | 92.8% |
| APHA Coloration | 42 |

### Example 14: Example Isosorbide Diester D2 Based on Isosorbide I2 According to Example 2

The esterification procedure and the purification techniques are identical to the preceding example, except that the starting isosorbide is 12.

The results are presented in Table 6.

TABLE 6

| Acid index | 1.5 mgKOH/g |
|---|---|
| Free fatty acid | 0.2% |
| Diester | 4.1% |
| Isosorbide | 93.3% |
| APHA Coloration | 82 |

From the results obtained in examples 13 and 14, isosorbide diesters based on isosorbide according to the invention have a more satisfactory coloring.

### Example 15: Example Isosorbide Diglycidyl Ether D3 Based on Isosorbide I1 According to Example 1

232 g of isosorbide, 644 g of epichlorohydrin (5 eq molar) and 2.32 g of tetraethylammonium bromide (TEAB) are introduced into a dual-jacketed stirred reactor equipped with a reverse Stark Dean surmounted by a condenser. The reaction medium is heated (setpoint temperature: 110° C.) at 275 mbar. After distillation of an amount of epichlorohydrin sufficient to fill the reverse Dean-Stark, 235 g of aqueous solution of sodium hydroxide at 50% by mass is introduced over a period of 3 hours using a pump. During the addition of sodium sulfate, the distillation of the water-epichlorohydrin azeotrope and the demixing in the Dean-Stark allow the water introduced and formed during the reaction to be removed. Once the addition of sodium sulfate is complete, the medium is allowed to warm and distill until the medium reaches a temperature of 90° C. Once this temperature is reached, heating is stopped and the medium is left to cool at ambient temperature. The medium is then stripped, and the salts formed during the reaction are filtered using a porosity 3 sintered glass. The salt cake is then washed using 150 g of epichlorohydrin. The filtrate is recovered. The residual epichlorohydrin is eliminated by distillation under vacuum using a rotary evaporator.

352 g of a yellow homogeneous viscous oil are obtained.

The results are presented in Table 7.

TABLE 7

| | |
|---|---|
| EPOXY equivalent (g/eq) | 175 |
| Isosorbide conversion rate (%) | 100% |
| Gardner Coloration | 1.6 |
| Viscosity (mPa · s) | 3100 |
| Residual epichlorohydrin (g/100 g) | Not detected |
| Water content (g/100 g) | 0.05 |

### Example 16: Example Isosorbide Diglycidyl Ether D4 Based on Isosorbide I2 According to Example 2

Reaction identical to the previous example except that the isosorbide used is I2. The results are presented in Table 8.

TABLE 8

| | |
|---|---|
| EPOXY equivalent (g/eq) | 178 |
| Isosorbide conversion rate (%) | 100% |
| Gardner Coloration | 2.7 |
| Viscosity (mPa · s) | 3400 |
| Residual epichlorohydrin (g/100 g) | Not detected |
| Water content (g/100 g) | 0.07 |

From the results obtained in examples 15 and 16, isosorbide diglycidyl ethers based on isosorbide according to the invention have a more satisfactory coloring.

### Example 17: Example Coating with Isosorbide Diglycidyl Ether D3 Based on Isosorbide I3 According to Example 3

5 g of isosorbide epoxy D3 are mixed with 1.18 g of IPDA (ANEW=42.5 g/eq). The mixture is then applied to a Q panel made of steel using a barcoater, and is then placed in an oven for 1 hour at 80° C. and then 2 hours at 180° C.

Final coating with a thickness of 151 microns has a Persoz hardness of 297s, a pencil hardness of 16N and a gloss of 96.7 at 20°. During the cross-cut adhesion test, no element is detached from the substrate.

### Example 18: Example Coating with Isosorbide Diglycidyl Ether D4 Based on Isosorbide I4 According to Example 4

5 g of isosorbide epoxy D4 are mixed with 1.18 g of IPDA (ANEW=42.5 g/eq). The mixture is then applied to a Q panel made of steel using a barcoater, and is then placed in an oven for 1 hour at 80° C. and then 2 hours at 180° C.

Final coating with a thickness of 145 microns has a Persoz hardness of 295s, a pencil hardness of 16N and a gloss of 91.1 at 20°. During the cross-cut adhesion test, no element is detached from the substrate.

From the results obtained in examples 17 and 18, isosorbide diglycidyl ether coatings based on isosorbide according to the invention have a more satisfactory gloss at 20° C.

1. A product of internal dehydration of sorbitol, wherein it has a total residual nitrogen atom content of between 0.01 ppm and 150 ppm, preferably of between 0.02 ppm and 20 ppm, more preferably of between 0.05 ppm and 10 ppm, and, more preferentially, of between 0.07 ppm and 5 ppm, this residual content being expressed as dry weight relative to the total dry weight of said product, and in that it has a total residual sulphur atom content of between 0.0001 ppm and 100 ppm, preferably between 0.0002 ppm and 50 ppm, more preferably between 0.0004 ppm and 30 ppm, and more preferentially, between 0.0008 ppm and 20 ppm, this total residual content being expressed in dry weight relative to the dry weight of said product.

2. The product according to claim 1, wherein it has a total residual content of sodium and potassium atoms comprised between 0.002 ppm and 100 ppm, preferably between 0.004 ppm and 50 ppm, more preferably between 0.006 ppm and 20 ppm, and, more preferentially, between 0.008 ppm and 10 ppm, this total residual content being expressed in dry weight relative to the total dry weight of said product.

3. The product according to claim 1, wherein it has a total residual content of calcium and magnesium atoms of between 0.005 ppm and 100 ppm, preferably between 0.010 ppm and 50 ppm, more preferably between 0.015 ppm and 20 ppm, and, more preferentially, between 0.020 ppm and 10 ppm, this total residual content being expressed in dry weight relative to the total dry weight of said product.

4. The product according to claim 1, wherein it has a total residual content of iron atoms of between 0.005 ppm and 100 ppm, preferably between 0.010 ppm and 50 ppm, more preferably between 0.015 ppm and 20 ppm, and, more preferentially, between 0.020 ppm and 10 ppm, this total residual content being expressed in dry weight relative to the total dry weight of said product.

5. The product according to claim 1, wherein it has a total residual content of chlorine atoms of between 0.005 ppm and 100 ppm, preferably between 0.010 ppm and 50, more preferably between 0.015 ppm and 20 ppm, and, more preferentially, between 0.020 ppm and 10 ppm, this total residual content being expressed in dry weight relative to the total dry weight of said product.

6. A method for purifying a product of internal dehydration of sorbitol according to claim 1, said method comprising a succession of steps:
a) a step of supplying said product of internal dehydration of sorbitol,
b) a step of distilling said dehydration product so as to form a distillation product A,
c) a step of dissolving in water said distillation product A with the addition of a basic compound so as to form a solution B,
d) at least one step of discoloration of solution B resulting from the step of redissolving with addition of a basic compound,
e) at least one step of ion exchange of the solution resulting from the bleaching step, and
f) a step of recovering the resulting purified product C,
said basic compound being added in an amount of between 1 and 6 g, preferably between 2 and 5 g per Kg of product of internal dehydration of sorbitol provided in step a).

7. The method according to claim 6, wherein the pH of the solution B is between 4 and 10, preferably between 7 and 9.

8. The method according to claim 6, wherein the basic compound is chosen from alkaline earth hydroxides such as magnesium hydroxide, calcium hydroxide, strontium hydroxide or barium hydroxide, preferably calcium hydroxide.

9. The method according to claim 6, wherein the treatment by the discoloration step comprises at least one passage over a column of granular activated carbon.

10. The method according to claim 6, wherein at least one ion exchange step is chosen from a passage on a cation exchange resin or a passage on an anion exchange resin or a mixture of two, preferably the cation exchange resin is a strong cation exchange resin and the anion exchange resin is a strong anion exchange resin.

11. The method according to claim 6, wherein the method is free of an additional discoloration step after the ion exchange step and before the step of recovering the resulting product.

12. A polymer selected from a polyester, a polycarbonate, a polyarylether, a polyurethane or a polyepoxide, said polymer is wherein it comprises a unit corresponding to the product of internal dehydration of sorbitol according to claim 1.

13. A polymer selected from a polyester, a polycarbonate, a polyarylether, a polyurethane or a polyepoxide, said polymer is wherein it comprises a unit corresponding to the product of internal dehydration of sorbitol obtained from a method according to claim 6.
